(19) United States
(12) Patent Application Publication
(10) Pub. No.: US 2009/0093700 A1
(43) Pub. Date: Apr. 9, 2009

(54) APPARATUS AND METHOD FOR MULTI-MODAL IMAGING USING NANOPARTICLE MULTI-MODAL IMAGING PROBES

(76) Inventors: Heidi Hall, Webster, NY (US); Mark Edward Bridges, Spencerport, NY (US); Silas J. Leavesley, Mobile, AL (US); Valeri P. Patsekin, Gatchina (RU); J. Paul Robinson, West Lafayette, IN (US); David L. Patton, Lebannon, PA (US); Douglas Lincoln Vizard, Durham, CT (US); William E. McLaughlin, Guilford, CT (US); Bartlomeiej P. Rajwa, West Lafayette, IN (US)

Correspondence Address: Susan L. Parulski;Patent Legal Staff, Carestream Health, Inc., 150 Verona Street, Rochester, NY, 14608, US

(21) Appl. No.: 12/201,204

(22) Filed: Aug. 29, 2008

Related U.S. Application Data
(63) Continuation-in-part of application No. 11/221,530, filed on Sep. 8, 2005.
(60) Provisional application No. 60/970,623, filed on Sep. 7, 2007.
(60) Provisional application No. 61/024,621, filed on Jan. 30, 2008.

(51) Int. Cl. A61B 5/05

(52) U.S. Cl. 600/407

(57) ABSTRACT

An apparatus for multimodal imaging of an object includes a support stage for receiving an object to be imaged; an object supported on the stage, a light source for producing a beam to illuminate the object; a filter positioned to receive and pass the beam toward the object; and a lens and camera system for capturing an image of the object. The apparatus may include a tiltable filter for filtering light from the source. The apparatus may include a mechanism for selectively directing light from the light source through a first filter assembly to produce a first beam of light of a first frequency range for illuminating an object on the stage in a first imaging mode or through a second filter assembly to produce a second beam of light of a second frequency range for illuminating an object on the stage in a second imaging mode, so that the lens and camera system captures light from the object illuminated by either the first or second beam of light to produce a first image in response to the first beam and a second image, different from the first image, in response to the second image. An x-ray source and phosphor plate may be included to provide an additional imaging mode.

### CROSS-REFERENCE TO RELATED APPLICATIONS

Priority is claimed from commonly assigned, copending provisional U.S. Patent Application Ser. No. 60/970,623 filed Sep. 7, 2007 by Harder et al, entitled “APPARATUS AND METHOD FOR MULTI-MODAL IMAGING USING NANOPARTICLE MULTI-MODAL IMAGING PROBES,” the disclosure of which is incorporated by reference in this specification. This application is a continuation-in-part of commonly assigned, copending (a) regular U.S. patent application Ser. No. 11/221,530 filed Sep. 8, 2005 by Vizard et al entitled “APPARATUS AND METHOD FOR MULTI-MODAL IMAGING”; and (b) provisional U.S. Patent Application Ser. No. 61/024,621 filed Jan. 30, 2008 by Feke et al entitled “APPARATUS AND METHOD FOR MULTI-MODAL IMAGING”, the disclosures of each of which are incorporated by reference in this specification.

### FIELD OF THE INVENTION

The invention relates generally to the field of imaging systems, and more particularly to the imaging of objects. More specifically, the invention relates to an apparatus and method enabling analytical imaging of objects (for example, small animals and tissue) in differing modes, including bright-field, dark-field (e.g., luminescence and fluorescence), x-ray and radioactive isotopes, and enhanced magnetic resonance imaging (MRI), by the use of injectable diagnostic agents for infrared and multimodal medical imaging.

### BACKGROUND OF THE INVENTION

Reference is made to commonly assigned, co-pending (a) regular U.S. patent application Ser. No. 11/400,935 (Docket 91687) filed Apr. 10, 2006 by Harder et al entitled “FUNCTIONALIZED POLY(ETHYLENE GLYCOL)”; (b) regular U.S. patent application Ser. No. 11/165,849 (Docket 88835CIP) filed Jun. 24, 2006 by Bringley et al entitled “NANOPARTICLE BASED SUBSTRATE FOR IMAGE CONTRAST AGENT FABRICATION”; and (c) regular U.S. patent application Ser. No. 11/872,866 (Docket 92735) filed Oct. 16, 2007 by Zheng et al entitled “ACTIVATABLE IMAGING PROBE USING NANOPARTICLES”, all of which are incorporated by reference in this application. Reference also is made to commonly assigned U.S. Pat. Nos. 6,444,988 and 7,031,084, which are incorporated by reference in this application.

Electronic imaging systems are well known for enabling molecular imaging. The electronic imaging system 10 shown in FIG. 1 and diagrammatically illustrated in FIG. 2 is the Image Station 4000MM manufactured by Carestream Health, Inc., that includes a light source 12, an optical compartment 14 which can include a mirror 16, a lens/camera system 18, and a communication/computer control system 20 which can include a display device, for example, a computer monitor. Camera/lens system 18 can include an emission filter wheel for fluorescent imaging. Light source 12 can include an excitation filter selector for fluorescent excitation or bright field color imaging. In operation, an image of an object is captured using lens/camera system 18. System 18 converts the light image into an electronic image, which can be digitized. The digitized image can be displayed on the display device, stored in memory, transmitted to a remote location, processed to enhance the image, and/or used to print a permanent copy of the image.

U.S. Pat. No. 6,495,812 discloses an apparatus used in the analysis of fluorescent markers attached to biological materials. In the apparatus disclosed the light source, a laser, and detector are mounted on the same movable device to allow the light beam and the focal point of the detector to intersect at the object of interest carried on a separate stage compensating for the variations in the thickness and material used to hold the sample on the stage. This device is limited to biological materials on a slide (e.g., strands of DNA). In addition the light source is of a fixed wavelength, that of the laser. U.S. Patent Publication No. 2004/0004193 discloses a fluorescent image capture device with a filter wheel disposed in the illumination path. The device disclosed is limited to the capture of only a fluorescent image. U.S. Patent Publication No. 2000/148846 discloses a fluorescent image capture device with a filter wheel disposed between the object being illuminated and the capture device. The device disclosed is limited to the capture of only a fluorescent image. U.S. Patent Publication No. 2005/0175538 discloses a device for collecting light emitted from an animal where a luminescent reporter has been injected into the animal. The device disclosed is limited to the capture of only a luminescent image.

To increase the effectiveness of these electronic imaging systems, efforts have been focused upon developing nanoparticulate systems capable of delivering imaging agents directly to the cells of interest. These nanoparticles can carry biological, pharmaceutical or diagnostic components within living systems. These nanoparticulate systems typically comprise drugs, therapeutics, diagnostics, biocompatibilization functionalities, contrast agents, and targeting moieties attached to or contained within a nanoparticulate carrier. Work in this field has the goals of affording imaging and therapeutic agents with such profound advantages as greater circulatory lifetimes, higher specificity, lower toxicity and greater therapeutic effectiveness. Work in the field of nanoparticulate assemblies has promised to significantly improve the treatment of cancers and other life threatening diseases and may revolutionize their clinical diagnosis and treatment.

Certain nanoparticles have been proposed as carriers for certain pharmaceutical agents. See for example: Sharma et al. Oncology Research 8, 281 (1996); Zobel et al. Antisense Nucl. Acid Drug Dev., 7:483 (1997); de Verdiere et al. Br. J. Cancer 76, 198 (1997); Hussein et al., Pharm. Res., 14, 613 (1997); Alyautdin et al. Pharm. Res. 14, 325 (1997); Hrkach et al., Biomaterials, 18, 27 (1997); Torchilin, J. Microencapsulation 15, 1 (1988); and literature cited therein.

The nanoparticle chemistries provide for a spectrum of rigid polymer structures, which are suitable for the encapsulation of drugs, drug delivery and controlled release. Some problems of these carriers include aggregation, colloidal instability under physiological conditions, low loading capacity, restricted control of the drug release kinetics, and synthetic preparations which are tedious and afford low yields of product.

The size of the nanoparticulate assemblies is one parameter determining their usefulness in biological compositions. After administration in the body, large particles are eliminated by the reticuloendothelial system and cannot be easily transported to the disease site (see for example, Volkheimer, Pathologe 14:247 (1993); Kwon and Kataoka, Adv. Drug. Del. Rev. 16:295 (1995).

Moghimi et al (Moghimi, S. M.; Hunter, A. C.; Murray, J. C. “Nanomedicine: Current Status and Future Prospects.” FASEB Journal 2005, 19, 311-330.) reports that particles larger than 100 nm are susceptible to clearance by interstitial macrophages while particles of 150 nm or larger are susceptible to accumulation in the liver. Also, the transport of large particles in the cell and intracellular delivery is limited or insignificant. See for example, Labhasetwar et al. Adv. Drug Del. Res. 24:63 (1997). It is suggested that an aggregated cationic species with a size from 500 nm to over 1 micron are ineffective in cell transfection. Large particles, particularly, those positively charged exhibit high toxicity in the body, in part due to adverse effects on liver and embolism. See for example Volkheimer, previously cited; Khopade et al Pharmazie 51:558 (1996); Yamashita et al., Vet. Hum. Toxicol, 39:71 (1997).

Specific nanoparticles have been found to be nontoxic, and are capable of entry into small capillaries in the body, transport in the body to a disease site, crossing biological barriers (including but not limited to the blood-brain barrier and intestinal epithelium), absorption into cell endocytic vesicles, crossing cell membranes and transportation to the target site inside the cell. The particles in that size range are believed to be more efficiently transferred across the arterial wall compared to larger size microparticles, see for example, Labhasetwar et al., previously cited. Without being bound by any particular theory, it may be that a high surface to volume ratio enables more successful targeting.

U.S. Patent Publication No. 2003/0211158 discloses microgels, microparticles, typically 0.1-10 microns in size, and related polymeric materials capable of delivering bioactive materials to cells for use as vaccines or therapeutic agents. The materials are made using a crosslinker molecule that contains a linkage cleavable under mild acidic conditions. The crosslinker molecule is exemplified by a bisacryloyl acetal crosslinker. The materials have the common characteristic of being able to degrade by acid hydrolysis under conditions commonly found within the endosomal or lysosomal compartments of cells thereby releasing their payload within the cell. The materials can also be used for the delivery of therapeutics to the acidic regions of tumors and sites of inflammation. These particles, however, are of a large enough size range that uptake by the reticuloendothelial system can be expected to be a problem. In addition, the degree of PEGylation is low and in-vivo agglomeration has been identified as a problem (see for example, Kwon, Y. J.; Standley, S. M.; Goh, S. L.; Frechet, J. M. J. Journal of Controlled Release 2005, 105, 199-212).

Some authors have described the difficulty of making stable dispersions of surface modified particles. Achieving stability under physiological conditions (e.g., pH 7.4 and 137 mM NaCl) is yet even more difficult. Burke and Barret (Langmuir, 19, 3297 (2003)) describe the adsorption of the amine-containing polyelectrolyte, polyallylamine hydrochloride, onto 70-100 nm silica particles in the presence of salt. The authors state (p. 3299) “the concentration of NaCl in the solutions was maintained at 1.0 mM because higher salt concentrations lead to flocculation of the suspension.”

WO 2004/108902 discloses using a biocompatible fluorescent nanoparticle imaging probe as part of a method using the Kodak 1D v.3.6.3 software (Kodak Imaging System) for dual modality imaging. The probes disclosed are single imaging probes, for example a fluorescent imaging probe and a different probe used for X-ray imaging. Rather than having both imaging modalities on one probe, the two different probes are injected into the subject at the same time or in quick succession.

Some fluorescent dyes have small separation between the energy of the absorption and emission, (often 10-30 nm at maximum commonly referred to as Stokes shift) so there is significant overlap in the spectral curves for absorption and emission. Filters can be used to insure that the excitation energy is not being detected during the measure of emission energy. The filters are often broad band pass filters (for example plus or minus 20 nm) or narrow band pass filters (for example plus or minus 10 nm). Because of this limitation, dyes are often excited at a higher energy than is optimum and the emission is measured at a lower energy than is optimum, which results in the need for more excitation energy from the light source and greater sensitivity from the detector or CCD. Some dyes have large bandwidths (>100 nm) and others have narrow bandwidths (<50 nm). For dyes with small Stokes shifts and narrow bandwidths, it is difficult to get efficient excitation and emission. Typical instruments have limited capability to choose the wavelength of excitation through the energy output of the light source and choice of the filter, which can create situations where fluorescent dyes cannot be used because too little energy is absorbed or too much energy must be filtered from emission. The combination of an instrument that will allow the variable selection of the excitation energy with fluorophores that absorb from 300 nm to 900 nm is desirable.

### SUMMARY OF THE INVENTION

An embodiment of an apparatus for multimodal imaging of an object in accordance with the invention may include a support stage for receiving an object to be imaged; an object supported on the stage, a light source for producing a beam to illuminate the object; a filter positioned to receive and pass the beam toward the object; and a lens and camera system for capturing a first image of the object. The filter may be tiltable relative to the beam and may be an interference filter or an acousto-optic tunable filter. A pair of AOTFs may be cascaded, whereby the beam is doubly filtered. An interference filter may be cascaded with an AOTF. In the latter embodiment, the interference filter may one in which tilt relative to the beam affects the spectral band that the interference filter passes and may further comprise means for tilting the filter relative to the beam.

Another embodiment of an apparatus for multimodal imaging of an object in accordance with the invention may include a light source; a support stage for receiving an object to be imaged; means for selectively directing light from the light source through a first filter assembly to produce a first beam of light of a first frequency range for illuminating an object on the stage in a first imaging mode or through a second filter assembly to produce a second beam of light of a second frequency range for illuminating an object on the stage in a second imaging mode; and a lens and camera system for capturing light from the object illuminated by either the first or second beam of light to produce a first image in response to the first beam and a second image, different from the first image, in response to the second beam. A third imaging mode may be provided by including an x-ray source and phosphor plate for producing light for capture by the camera and lens system.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 shows a perspective view of an exemplary electronic imaging system.

FIG. 2 shows a diagrammatic view of the electronic imaging system of FIG. 1.

FIG. 3A shows a diagrammatic side view of an imaging system useful in accordance with the present invention.

FIG. 3B shows a diagrammatic front view of the imaging system of FIG. 3A.

FIG. 4 shows a perspective view of the imaging system of FIGS. 3A and 3B.

FIG. 5A shows a diagrammatic side view of a sample object stage useful in accordance with the invention.

FIG. 5B shows a diagrammatic side view of the sample object stage in the first imaging position P1 wherein the phosphor plate is disposed proximate the sample object stage.

FIG. 5C shows a diagrammatic side view of the sample object stage in the second imaging position P2 wherein the phosphor plate is not proximate the sample object stage.

FIG. 6 shows an enlarged, fragmentary sectional side view taken along line 6-6 of FIG. 5B.

FIG. 7 shows an enlarged, fragmentary sectional side view taken along line 7-7 of FIG. 5C.

FIG. 8A shows a schematic view of a multi-spectral light source with a slideably and tiltably mounted filter assembly in accordance with the invention.

FIG. 8B shows a schematic view of the slideably and tiltably mounted filter pack of the multispectral light source in accordance with the invention.

FIG. 8C shows schematic plan and side views of one embodiment of a rotatably and tiltably mounted filter assembly of the multispectral light source, in accordance with the invention.

FIG. 9A shows a schematic view of a multispectral light source with an acousto-optic tunable filter (AOTF) in accordance with the invention.

FIG. 9B illustrates the AOTF diffracted beam angular configuration.

FIG. 9C shows a system overview flowchart for an excitation-scanning hyper-spectral imager in accordance with the invention.

FIG. 9D shows a schematic view of a multispectral light source with a slideable filter assembly, an acousto-optic tunable filter (AOTF), and a movable light path control assembly in position “A” in accordance with the invention.

FIG. 9E shows a schematic view of a multispectral light source with slideable filter assembly, an acousto-optic tunable filter (AOTF), and a movable light path control assembly in position “B” in accordance with the invention.

FIG. 9F shows a schematic view of a multispectral light source with slideable filter assembly and an acousto-optic tunable filter (AOTF) in a cascaded configuration.

FIG. 9G shows a schematic view of a multispectral light source with two acousto-optic tunable filters (AOTFs) in a cascaded configuration.

FIG. 10 shows a schematic view of a rotatably mounted filter pack of bandpass filters in accordance with the invention.

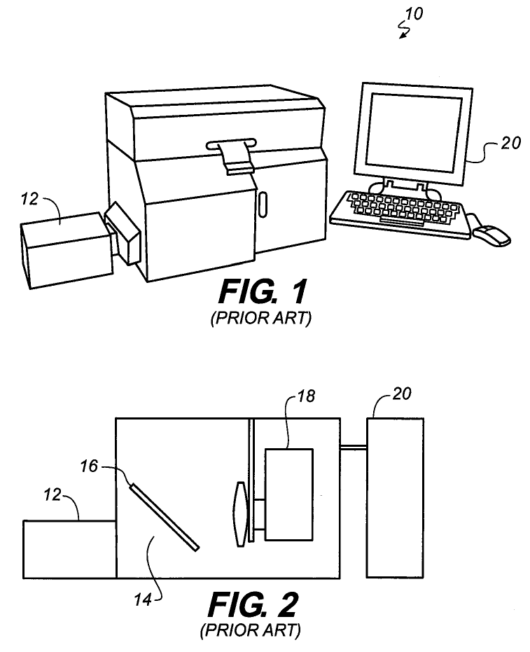

### DETAILED DESCRIPTION OF THE INVENTION

The invention will described in detail with particular reference to certain embodiments thereof, but it will be understood that variations and modifications can be effected within the spirit and scope of the invention.

In the complex pharmaceutical analyses of images of small objects or subjects, such as small animals and small volumes of tissue in larger animals, it is advantageous to obtain images, which are particularly enhanced by using different in-vivo, imaging modalities. Using the current practices of bright-field, dark-field and radiographic imaging for the analysis of small objects or subjects (such as a mouse) can be expensive and may not provide the precision of co-registered images that is desired.

By treating the animals or tissues with imaging probes which are configured to be the multimodal biological targeting units and using an apparatus and a method of the present invention, precisely co-registered dual modality imaging units comprised of nanoparticles within a subject animal or tissue can be localized; and multiple images from various imaging modalities can be obtained and accurately overlaid onto a simple bright-field reflected image of the same animal or tissue within minutes of animal immobilization or placement of a tissue sample.

The present invention uses an integrated imaging system to capture images using differing imaging modes including multi-spectral illumination, thereby enabling simplified multi-modal imaging. More particularly, using the imaging system of the present invention, an immobilized object, such as an animal or tissue sample, can be imaged in several imaging modes without changing or moving the immobilized object. These acquired multi-modal images can then be merged to provide one or more co-registered images for analysis.

Imaging modes supported by the method, apparatus and probes of the present invention include: x-ray imaging, bright-field imaging, dark-field imaging (including luminescence imaging, fluorescence imaging) and radioactive isotope imaging. Images acquired in these modes can be merged in various combinations for analysis. For example, an x-ray image of the object can be merged with a near infrared (NIR) fluorescence image of the object to provide a new image for analysis.

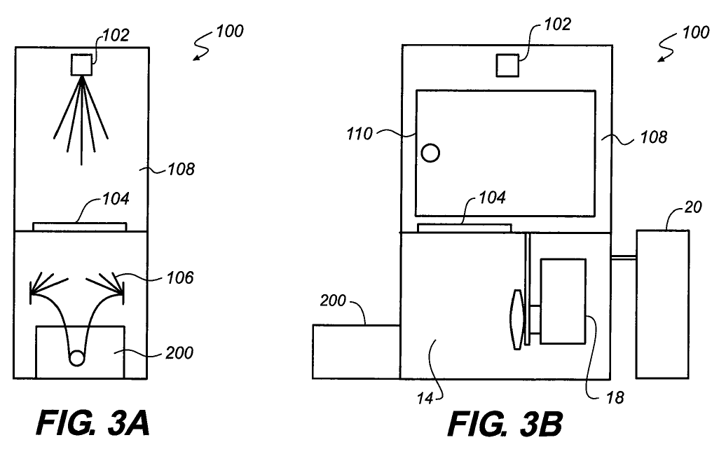

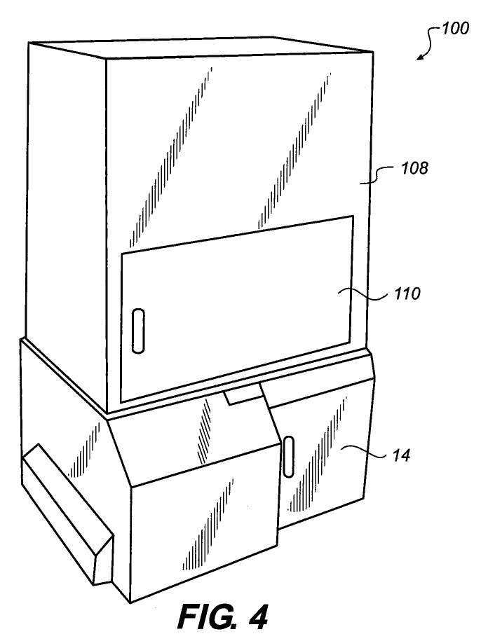

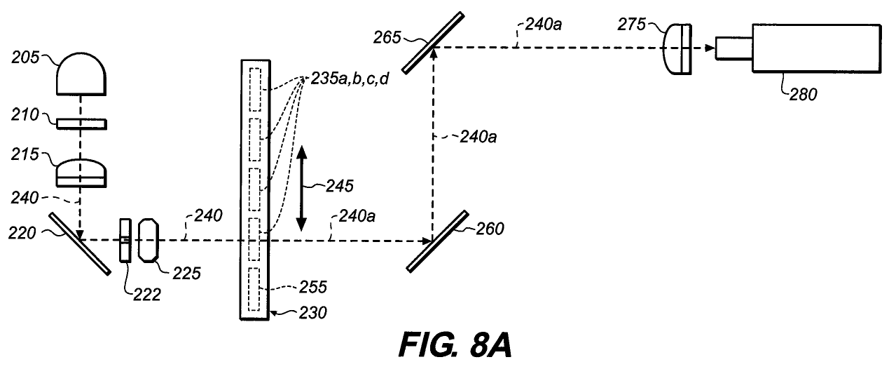

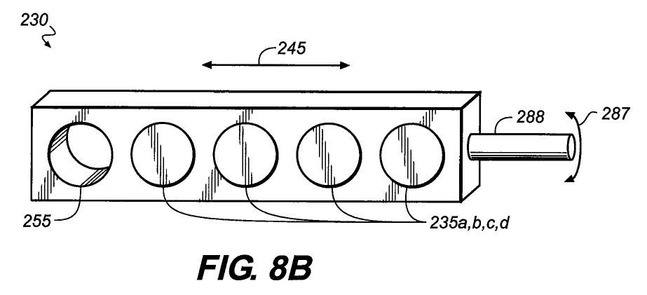

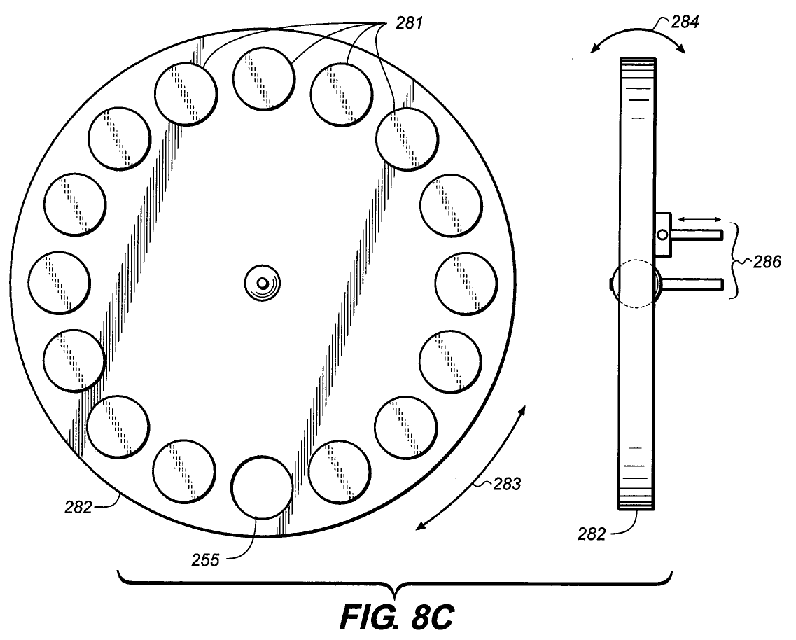

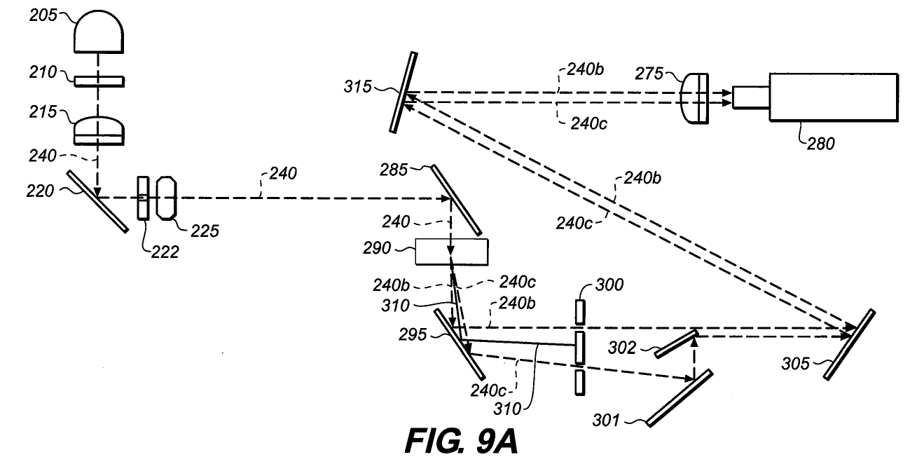

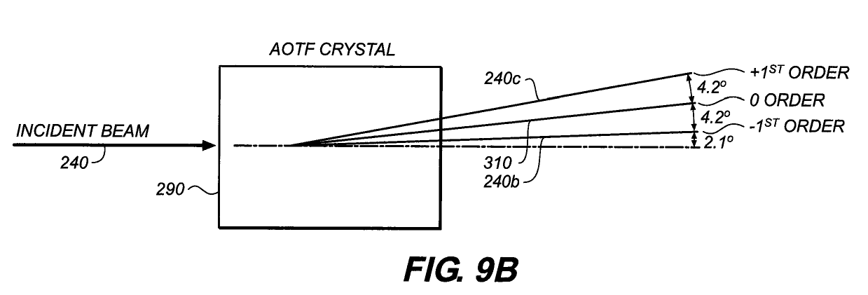

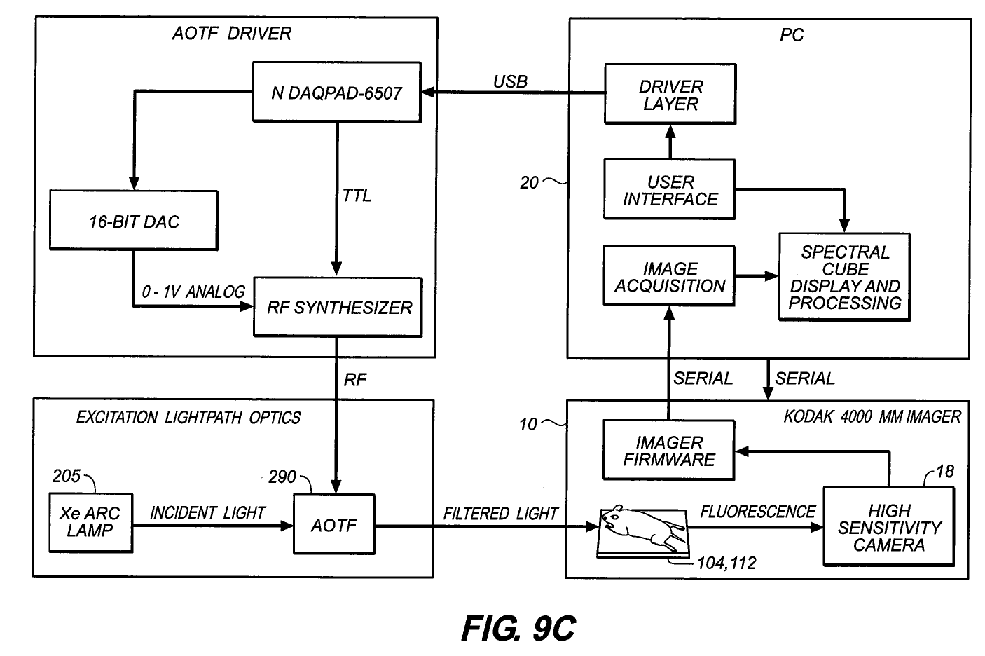

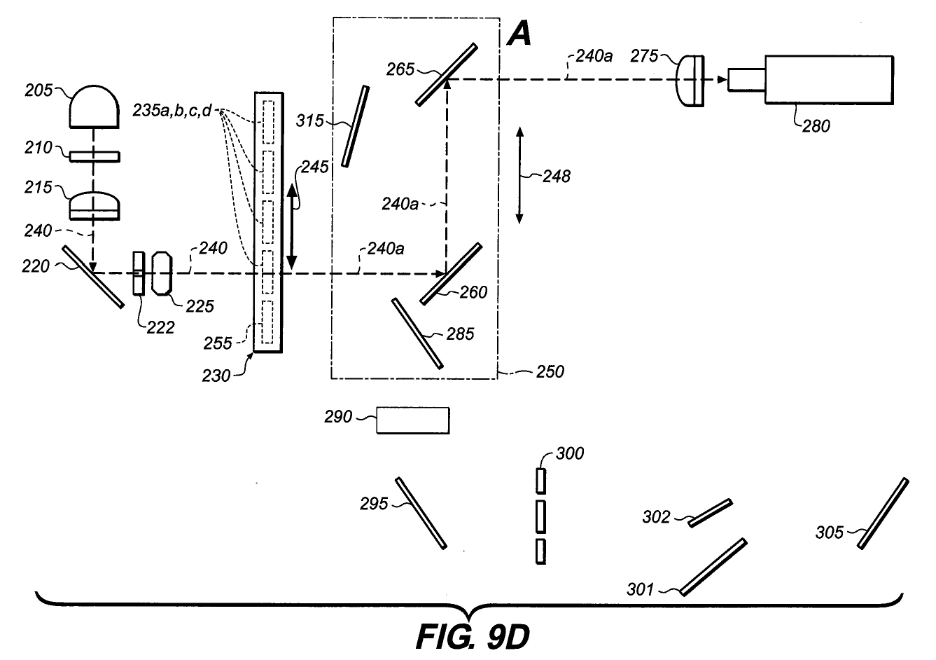

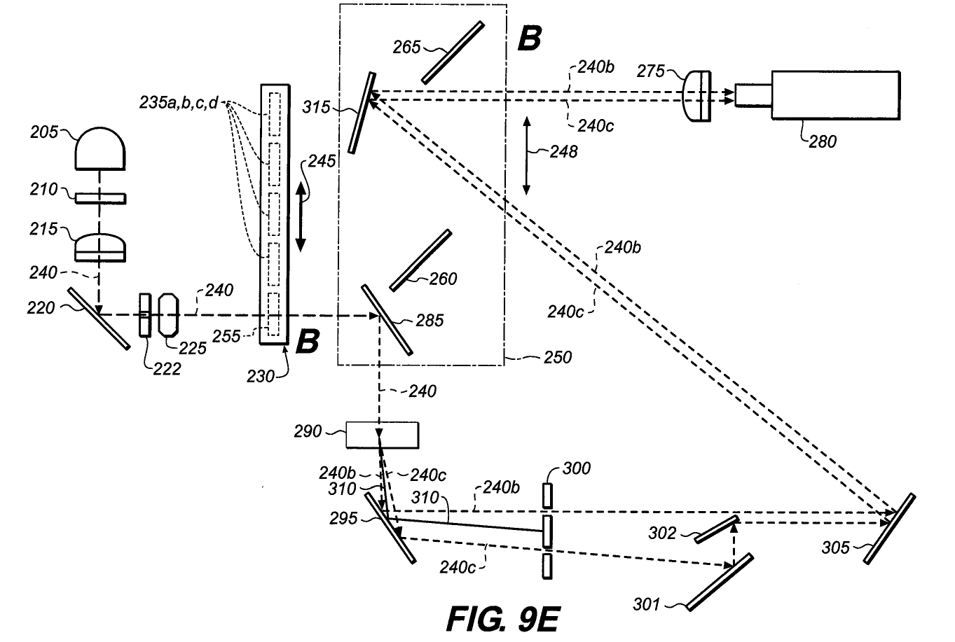

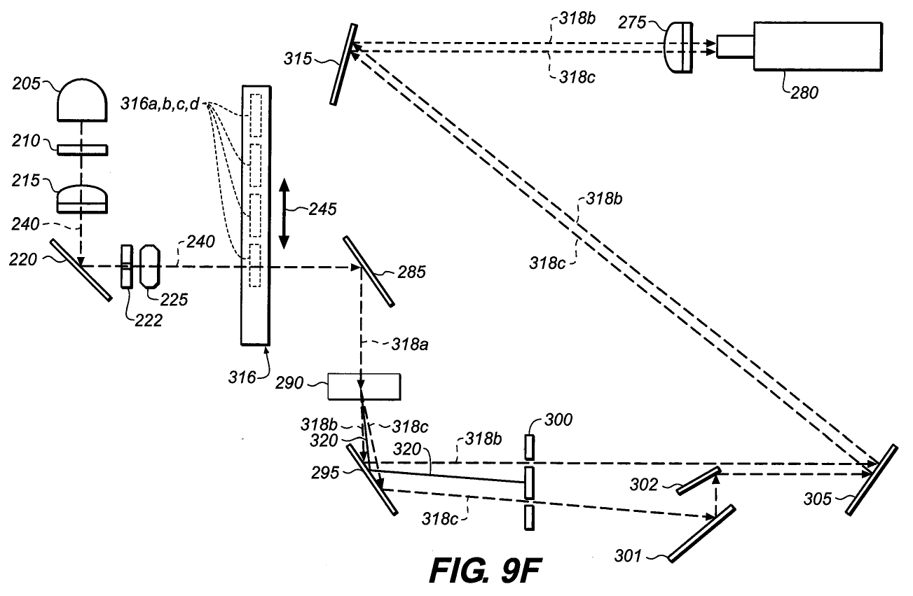

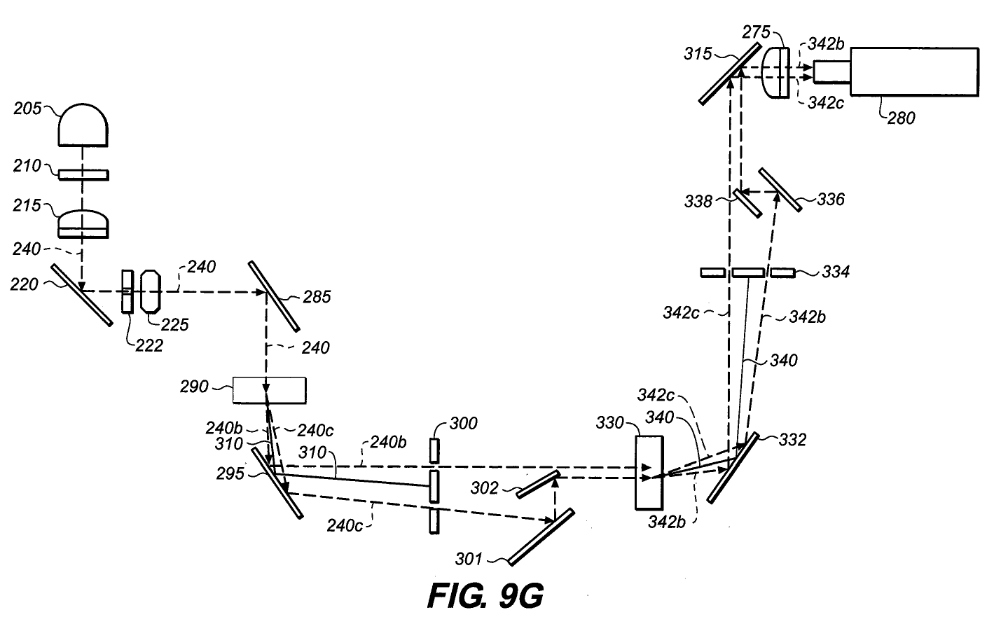

An apparatus for multi-modal imaging, suited for use in accordance with the present invention, is shown in FIGS. 3A, 3B, and 4. FIG. 3A shows a diagrammatic side view of an imaging system 100 that includes a programmable multispectral light source 200 (described in more detail with respect to FIGS. 8A and 9A), optical compartment 14, a lens/camera system 18, and communication/computer control system 20 which can include a display device, such as a computer monitor. Camera/lens system 18 can include an emission filter wheel for fluorescent imaging. Imaging system 100 may include an imaging means comprising an x-ray source 102 and a sample object support stage 104 to receive an object to be imaged. Imaging system 100 also may include a further imaging means comprising epi-illumination, for example, using fiber optics 106, which direct conditioned light (of appropriate wavelength and divergence as described with respect to FIGS. 8A and 9A) toward sample object stage 104 to provide bright-field or fluorescent imaging. Those skilled in the art will understand that “epi-illumination” refers to illumination and detection from one side of the object, as compared to trans-illumination. Sample object stage 104 is disposed within a sample environment 108, which allows access to the object being imaged. For example, sample environment 108 may be light-tight and fitted with light-locked gas ports, not illustrated, for environmental control. Environmental control enables practical x-ray contrast below 8 Kev (air absorption) and aids in life support for biological specimens. An access means or member 110 may be included to provide convenient, safe and light-tight access to sample environment 108, such as a door, opening, labyrinth, and the like. Additionally, sample environment 108 may be adapted to provide atmospheric control for sample maintenance or soft x-ray transmission, such as temperature and humidity controls, sources of alternative ambient gases and the like.

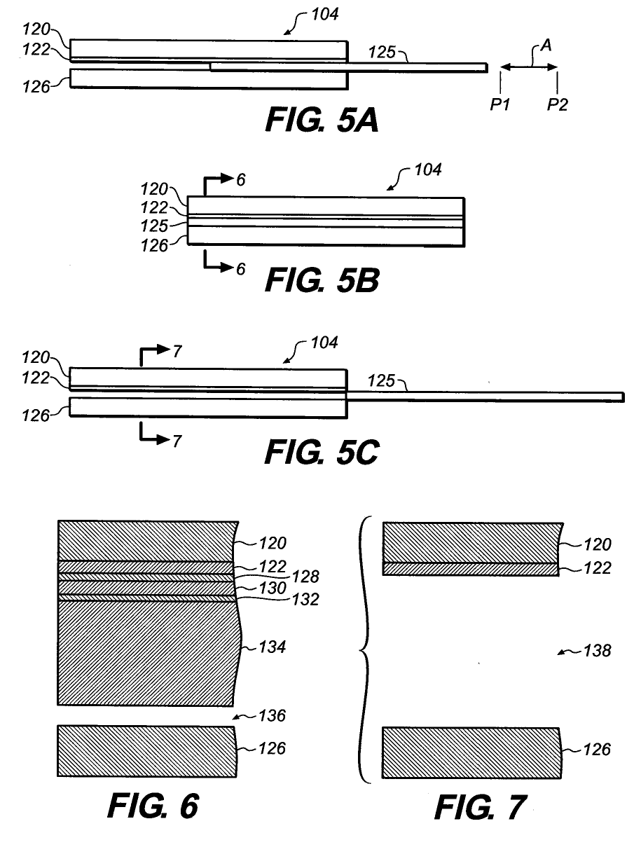

FIGS. 5-7 more particularly illustrate elements of sample object stage 104 and an optical interface relative with the focal plane of camera and lens system 18. FIG. 5A shows a diagrammatic side view of sample object stage 104 showing the relative movement of a phosphor plate 125 relative to the sample object stage. FIG. 5B shows a diagrammatic side view of the sample object stage in a first imaging position P1 wherein phosphor plate 125 is disposed proximate the sample object stage. FIG. 5C shows a diagrammatic side view of the sample object stage in the second imaging position P2 wherein phosphor plate 125 has been withdrawn to a position not proximate the sample object stage. FIG. 6 shows an enlarged sectional view that corresponds with the first imaging position P1. FIG. 7 shows an enlarged sectional view that corresponds with the second imaging position P2.

Continuing with regard to FIGS. 5 to 7, sample object stage 104 includes a support member made up from an open rectangular frame 120 on which is stretched a thin plastic support sheet 122. Support sheet 122 is selected so as to support the weight of an object to be imaged (not illustrated) and is made from a material that is optically clear and free of significant interfering fluorescence.

Phosphor plate 125 is mounted slideably for motion toward and away from sample object stage 104, such as on guide rails or rollers, not illustrated. While those skilled in the art might recognize other configurations, in one embodiment, phosphor plate 125 is mounted for sliding translation in the direction of arrow A relative to frame 120, beneath the sample, and in intimate contact with the underside of support sheet 122, as illustrated. As will be more particularly described below, in first imaging position P1 shown in FIG. 5B, phosphor plate 125 is in position directly beneath, opposite and proximate to sample object stage 104. In position P1, an x-ray image of the object is captured. In second imaging position P2 shown in FIG. 5C, phosphor plate 125 is translated or moved away from sample object stage 104. In position P2, capture of an image of the object can be achieved while phosphor plate 125 is not imaged.

FIG. 6 provides an enlarged, sectional view of sample object stage 104, with phosphor plate 125 in position P1 to more particularly show a focal plane at the bottom phosphor layer of plate 125. Sample support sheet 122 may be comprised of Mylar or polycarbonate, having for example, a nominal thickness of about 0.1 mm. A protective layer 128, such as reflective Mylar, of about 0.025 mm thickness may be provided to protect the phosphor surface of phosphor plate 125 during movement past support sheet 122. Protective layer 128 also may promote or increase the image-forming light output. In one embodiment, protective layer 128 is reflective so as to prevent object reflection back into the image-forming screen, reducing confusion of the ionizing radiation image.

Plate 125 further comprises a phosphor layer 130 that transduces ionizing radiation to visible light practically captured and managed by a lens and a camera system 18, such as a CCD camera. Phosphor layer 130 can have a thickness ranging from about 0.01 mm to about 0.1 mm, depending upon the application (i.e., soft x-ray, gamma-ray or fast electron imaging). On the underside of phosphor layer 130, as illustrated, an optical layer 132 is provided for conditioning emitted light from phosphor layer 130. Optical layer 132 can have a thickness in the range of less than about 0.001 mm. Particular information about phosphor layer 130 and optical layer 132 is disclosed in commonly assigned U.S. Pat. No. 6,444,988. A supporting glass plate 134 is provided for plate 125. Glass plate 134 is spaced at a suitable mechanical clearance from an optical platen 126, for example, by an air gap/void 136. In one embodiment, the surfaces of clear optical media (e.g., a lower surface of glass plate 134 and both surfaces of optical platen 126) are provided with anti-reflective coating to minimize reflections that may confuse the image of the object. FIG. 7 provides an enlarged sectional view of sample object stage 104 with phosphor plate 125 in position P2 where it is fully removed from the object stage, leaving frame 120, sample support sheet 122, and an air gap or void 138 between object stage 104 and optical platen 126.

In the multi-spectral light source shown in FIG. 8A, a bandpass interference filter assembly 230 is used to control the wavelength and the path of the light for illumination of the subject or object 112 on the sample object stage 104. The position of assembly 230 may be selectively, automatically controlled by communication/computer control system 20 as indicated by arrow 245. In the particular filter assembly embodiment shown in FIG. 8A, assembly 230 is depicted as a slide that also is tiltable on an axle 288 relative to the beam of light as indicated in FIG. 8B by arrow 287. Any suitable means may be used to tilt assembly 230, such as a servo-motor, not illustrated, for rotating shaft 288. Assembly 230 may be slideably mounted by techniques familiar to those skilled in the mechanical arts, such as slides or rollers. Assembly 230 houses a plurality of different bandpass interference filters 235a, b, c, and d designed to pass specific spectral bands of light, and a blank hole 255. In the filter assembly embodiment shown in FIG. 8C, multiple bandpass interference filters 281 are mounted on a rotatable wheel 282 capable of moving as indicated by arrow 283. Wheel 282 also may be tilted by a suitable mechanism 286 as indicated by arrow 284. The number of bandpass interference filters mounted on wheel 282 can vary depending on how broad a spectrum of wavelengths one wishes to cover and how finely one wants to segment the spectrum. The tilt of the filter assembly 230 or filter wheel 282 can be adjusted to affect the angle of incidence of the light striking the selected bandpass interference filter. By adjusting the angle of incidence, the efficiency of the bandpass interference filter to pass a desirable portion of the illumination striking the object on stage 104 can be greatly increased or the undesirable portion may be greatly decreased. In the example shown in FIG. 8C, there are 15 bandpass interference filters 281 and a blank hole 255 in the 16^{th} position.

Referring again to FIG. 8A, light source 200 of FIGS. 3A and 3B includes a xenon lamp 205 that directs a beam of light 240 in the range of 350 to 1100 nm through a series of optical elements including a hot mirror 210, which limits the wavelengths to the range of 400 to 800 nm, and a beam expander lens 215 or other light beam conditioning component, to a fold mirror 220 and through an aperture 222 (commonly referred to as a spatial filter), a collimating lens 225, and then to bandpass interference filter assembly 230, whose tilt can be adjusted as previously mentioned to affect the angle of incidence of light striking the bandpass interference filter. By adjusting the angle of incidence, the efficiency of the bandpass interference filter to pass a desirable portion of the illumination striking the subject can be greatly increased or the undesirable portion may be greatly decreased. The focal lengths of lenses 215 and 225 are chosen depending on the degree of collimation of light desired, size of the source aperture and size of the bandpass interference filters desired.

Communication/computer control system 20 positions appropriate bandpass interference filter 235a, b, c, or d into the path of light beam 240 depending on what spectral band of light is chosen for a first beam to illuminate the subject/object 112 on the sample object stage 104. The wavelengths of the bandpass interference filters in bandpass interference filter assembly 230 can range from 400 to 800 nm, each having a spectral bandpass of between 10 to 20 nm, for example. Portions of the spectral bandpass of the filters can overlap if desired. Light beam 240a, now filtered to the desired spectral band, is directed via a first angled mirror 260 and a second angled mirror 265 to lens 275, which focuses light beam 240a onto an optical fiber input 280 that transmits the light to illuminate a the subject or object 112 on the sample object stage 104 (as shown schematically in FIG. 9C).

Acousto-optic tunable filters (AOTF) have previously been investigated as alternatives to spectrometers and for spectral selection of fluorescent emission, but have not been used or proposed as a method or device to selectively tune within a wide wavelength range of excitation light, for example from a xenon lamp, for fluorescence imaging. The following description concerns an embodiment of an illumination system suitable for exciting fluorescent contrast agents for optical molecular imaging.

Now referring to FIGS. 9A to 9C, an AOTF 290 is used to control the wavelength of the light from xenon lamp 205 to illuminate the subject/object 112 on sample object stage 104. AOTF 290 may be, for example, a NEOSNEOS Technologies 48097-10-0.6 AOTF utilizing a TeO₂ crystal, chosen to select a narrow spectral band between 450 to 750 nm having a through range of between 7 to 35 nm and a diffraction efficiency of greater than 80% over the tuning range with an active aperture of the device of 10 mm×10 mm. AOTF 290 is used as an alternative to the interference filter assembly 230 described with regard to FIG. 8A. Accurate alignment of the AOTF crystal to the incident beam, such that the input beam is normal to the input face, is preferable so that the spectral bands will match for the two diffracted beams, namely the −1^{st} diffracted order and the +1^{st} diffracted order beams. The NEOS Technologies AOTF was designed so that any linearly-polarized component of input light inside the selected spectral band that is vertical with respect to the AOTF crystal and therefore diffracted into the −1^{st} diffraction order, with a 90° rotation of polarization on the output, is deflected 2.1° relative to the input beam whereby the deflection angle is constant over the tuning range of the device. Also, in such an AOTF; any linearly-polarized component of input light inside the selected spectral band that is horizontal with respect to the AOTF crystal and therefore diffracted into the +1^{st} diffracted order, with a 90° rotation of polarization on the output, is typically deflected 10.5° relative to the input beam whereby the deflection angle is slightly variable over the tuning range of the device. Also, any input light outside the selected spectral band remains undiffracted in the 0^{th} order beam which is typically deflected 6.3° relative to the input beam whereby the deflection angle is slightly variable over the tuning range of the device, hence the undiffracted 0^{th} order beam is typically deflected 4.2° relative to both the −1^{st} and +1^{st} diffracted order beams, as shown in FIG. 9B. The width of the spectral band also varies with the central wavelength of the spectral band, as shown in Table 1.

TABLE 1
Variation in AOTF spectral band width with variation of spectral band
central wavelength.

| Central | Spectral | Acoustic |
|---|---|---|
| Wavelength | Bandwidth | Frequency |
| 450 nm | 8.5 nm | 128.6 MHz |
| 550 nm | 15.0 nm | 97.0 MHz |
| 650 nm | 25.0 nm | 78.8 MHz |
| 750 nm | 37.0 nm | 66.7 MHz |

Because the light generated by xenon lamps is substantially unpolarized, the light from the xenon lamp input to the AOTF crystal in the selected spectral band results in both the +1^{st} diffracted order and −1^{st} diffracted order beams being output. Both the −1^{st} diffracted order and the +1^{st} diffracted order beams can be used by recombining them after the undiffracted 0^{th} order beam is dumped or trapped, thus providing the advantage of maximal light intensity in the selected spectral band relayed to the object/subject as needed for high-speed fluorescence imaging. Alternatively, the input light may be linearly polarized either horizontally or vertically with respect to the AOTF crystal, for example by a polarizer, if only a simplified optical relay geometry is desired; in this case, vertical linearly-polarized light would be desirable due to the constant deflection angle over the tuning range of the device which enables the most simplified optical relay geometry.

For this embodiment, optimum spectral purity of the tuned light was desired. In order to maximize spectral purity, the −1^{st} diffracted order and +1^{st} diffracted order beams, which contain the light in the selected spectral band, must be spatially separated from the undiffracted 0^{th} order beam that contains the light outside the selected spectral band. The maximum allowable divergence (full angle) was determined by the diffraction angle of the AOTF as Δθi=Δθ. If Δi is larger than Δθ, the diffracted and undiffracted beams will overlap and efficient separation will be impossible. All three of the output beams have the same divergence as the input beam up to the acceptance angle of the crystal, which in this exemplary system was greater than 8°. In order to separate the undiffracted 0^{th} order beam from the −1^{st} and +1^{st} diffracted order beams, the divergence half-angle of the input light preferably should be no greater than half the separation angle between the undiffracted 0^{th} order beam and the −1^{st} and +1^{st} diffracted order beams. The beams begin to diffract and separate angularly shortly before exiting the crystal. Depending on the cross-sectional area and the divergence of the beams, the beams need to propagate some distance before the undiffracted 1^{th} order beam and the −1^{st} and +1^{st} diffracted order beams are spatially separated. To ensure that the divergence half-angle of the input light will be no greater than half the separation angle between the undiffracted 0^{th} order beam and the −1^{st} and +1^{st} diffracted order beams, first the separation angle of the undiffracted 0^{th} order beam is maximized by appropriate selection of the AOTF, and then as necessary the divergence of the source is reduced. For example, a xenon lamp having a divergence half-angle of 4° and an AOTF having a separation angle of 4.2°, hence half of the separation angle 2.1°, between the undiffracted 0^{th} order beam and the −1^{st} and +1^{st} diffracted order beams requires the divergence of the light from the xenon lamp needs to be reduced before entering the AOTF. This can be achieved by using a pair of lenses 215 and 225 with a spatial filter 222 at their common focus. The diameter of the spatial filter in combination with the ratio of the focal lengths of the lenses may be used to select the half-angle of the divergence of the resulting beam, preferably to be only slightly less than half of the separation angle between the undiffracted 0^{th} order beam and the −1^{st} and +1^{st} diffracted order beams to avoid excessive rejection of useful light input to the AOTF.

Furthermore, it is often desirable to reduce Δθi as λ is a function of Δθi as shown in the equation

$$λ = \frac{v Δ n}{f} \sqrt{{Sin}^{4} {θ}_{i} + {Sin}^{4} {θ}_{i}}$$

meaning that a Δθi larger than several degrees will result in a range of λ, or equivalently, a larger bandwidth as shown in the equation

$$Δλ = \frac{0.9 {λ}^{2}}{Δ nL {sin}^{2} {θ}_{i}}$$

In small-animal fluorescence imaging, spectral resolution is not weighted as highly as illumination power or IBOBR defined as in-band to out-of-band ratio, which characterizes the quality of the illumination spectrum, and thus the spatial filter diameter is narrowed only enough to achieve spatial separation of the diffracted beams and no further. The output beam from the xenon lamp is for example 25 mm in diameter. In order to pass as much light as possible through the AOTF, the entire 10 mm×10 mm active aperture of the AOTF is used.

Referring again to FIG. 9A, a xenon lamp 205 directs a beam of light 240 in the range of 350 to 1100 nm through a series of optical elements including a hot mirror 210, which limits the wavelengths to the range of 400 to 800 nm, a beam expander lens 215 to a fold mirror 220 and through an aperture 222 (commonly referred to as a spatial filter), a collimating lens 225 to a third angled mirror 285 into the AOTF 290. The undiffracted 0^{th} order light beam 310, and the −1^{st} and +1^{st} diffracted order light beams 240b and 240c which have been filtered by AOTF 290 to the desired spectral band, are directed via a fourth angled mirror 295 to a baffling wall 300 used to eliminate the light beam 310 outside the selected spectral band from light beams 240b and 240c inside the selected spectral band. Light beam 240c is redirected by a fifth angled mirror 301 and an sixth angled mirror 302 to be collocated with light beam 240b. Light beams 240b and 240c are now directed by a seventh angled mirror 305 to an eighth angled mirror 315, through lens 275, and onto optical fiber input 280.

Referring now to the flow chart shown in FIG. 9C, communication/computer control system 20 (FIG. 3B) of the Image Station 4000MM (Carestream Health, Inc., Rochester, N.Y.) contains software that controls both the AOTF frequency driver and the Image Station 4000MM via a user interface and a driver layer. The user interface also controls display, manipulation, processing, and classification of the acquired spectral image data. The AOTF frequency driver consists of a radio-frequency (rf) sine wave generator, power supplies, and supporting electronics.

The configuration implemented in the embodiment of FIGS. 9A to 9C achieves an IBOBR of 20.1 and is able to discriminate among three subdermally injected fluorescent dyes and mouse autofluorescence. In another embodiment two or more AOTFs may be cascaded (not illustrated) for better control for selecting light inside the desired spectral band and rejecting light outside the desired spectral band. In another embodiment an interference filter and an AOTF may be cascaded (not illustrated) for better control for selecting light inside the desired spectral band and rejecting light outside the desired spectral band. In another embodiment, one or more interference filters and one or more AOTFs may be cascaded (not illustrated) for better control for selecting light inside the desired spectral band and rejecting light outside the desired spectral band.

In another embodiment shown in FIGS. 9D and 9E, a moveable assembly 250 permits the use of either the bandpass interference filter assembly 230 or the AOTF 290 or both in sequence. With moveable assembly 250 located in position “A” of FIG. 9D, a xenon lamp 205 directs a beam of light 240 through the hot mirror 210, the lens 215 or other light beam conditioning component, to the fold mirror 220 and through the aperture 222 (commonly referred to as a spatial filter), the collimating lens 225, then to bandpass interference filter assembly 230, whose tilt can be adjusted to affect the angle of incidence of light striking the bandpass interference filter as previously discussed. Filter assembly 230, shown in FIG. 9D, is slideably and tiltably mounted and houses the plurality of different bandpass interference filters 235a, b, c, and d designed to pass specific wavelengths of light, and a blank hole 255.

Communication/computer control system 20 positions appropriate filter 235a, b, c, or d into the path of light beam 240 depending on what wavelength of light is chosen to illuminate the subject/object 112 on the sample object stage 104. Light beam 240a now filtered to the desired spectral band is directed via the first angled mirror 260 and the second angled mirror 265 both mounted on the moveable assembly 250. Light beam 240a is then directed through a lens 275 and onto an optical fiber input 280 of the imaging system 10.

With movable assembly 250 located in position “B” of FIG. 9E, the AOTF 290, as previously discussed is used to control the wavelength of the light from xenon lamp 205 for illumination of the subject/object 112 on sample object stage 104. When AOTF 290, controlled by communication/computer control system 20, is used, blank hole 255 on filter assembly 230 is positioned in the path of light beam 240 and movable assembly 250 is moved to position “B”. With movable assembly 250 in position “B”, light beam 240 follows the path previously described and passes through blank hole 255 and is directed by a third angled mirror 285 mounted on movable assembly 250 into AOTF 290. The undiffracted 1^{th} order light beam 310, and the −1^{st} and +1^{st} diffracted order light beams 240b and 240c which have been filtered by AOTF 290 to the desired spectral band, are directed via a fourth angled mirror 295 to a baffling wall 300 used to eliminate the light beam 310 outside the selected spectral band from light beams 240b and 240c inside the selected spectral band. Light beam 240c is redirected by a fifth angled mirror 301 and an sixth angled mirror 302 to be collocated with light beam 240b. Light beams 240b and 240c are now directed by a seventh angled mirror 305 to an eighth angled mirror 315, through lens 275, and onto optical fiber input 280. In yet another embodiment, one of filters 235a, b, c, d on filter assembly 230 may be used in conjunction with the AOTF 290, instead of blank hole 255, with the moveable assembly 250 located in the “B” position.

In the embodiment shown in FIG. 9F, a multispectral light source includes a slideable low-pass interference filter assembly similar to that shown in FIG. 8A, but now in a cascaded configuration with an AOTF of the type shown in FIG. 9A. Particularly, angled mirrors 260 and 265 of the embodiment of FIG. 8A have been replaced with angled mirror 285, AOTF 290, angled mirror 295, baffling walls 300 and angled mirrors 301, 302, 305 and 315 of the embodiment of FIG. 9A. In the FIG. 9F embodiment, filter assembly 230 of FIG. 8A has been replaced with a slideable, low-pass interference filter assembly 316 that includes a plurality of different low-pass interference filters 316a, b, c and d designed to pass specific spectral bands of light. Filters 316a to 316d alternatively could be provided on a rotatable wheel such as in FIG. 8C. In this embodiment, tilting of filter assembly 316 is not required since tuning is provided by downstream AOTF 290. In the manner previously described, communication and computer control system 20 positions appropriate low-pass filter 316a, b, c or d into the path of light beam 240, depending on what spectral band of light is chosen. A low-pass filtered light beam 318 thus issues from filter assembly 316 and is reflected by angled mirror 285 into AOTF 290. The undiffracted 1^{th} order low-pass filtered light beam 320, and the −1^{st} and +1^{st} diffracted order doubly filtered light beams 318b and 318c which have been filtered by AOTF 290 to the desired spectral band, are directed via angled mirror 295 to a baffling wall 300 used to eliminate the light beam 320 outside the selected spectral band from light beams 318b and 318c inside the selected spectral band. Light beam 318c is redirected by angled mirror 301 and angled mirror 302 to be collocated with light beam 318b. Light beams 318b and 318c are now directed by angled mirror 305 to angled mirror 315, through lens 275, and onto optical fiber input 280.

In the embodiment shown in FIG. 9G, a multispectral light source includes a first AOTF of the type shown in FIG. 9A, but now in a cascaded configuration with a second AOTF of the same type. Particularly, angled mirror 305 of the embodiment of FIG. 9A has been replaced, in series, with a second AOTF 330, an angled mirror 332, a baffling wall 334, and angled mirrors 336 and 338. Thus, downstream of angled mirrors 301 and 302, filtered light beams 240b and 240c enter AOTF 330. At the output of AOTF 330, the residual undiffracted 1^{th} order light beam 340, and the −1^{st} and +1^{st} diffracted order light beams 342b and 342c which have been filtered by AOTF 330 to the desired spectral band, are directed via angled mirror 332 to baffling wall 334 used to eliminate the light beam 340 outside the selected spectral band from light beams 342b and 342c inside the selected spectral band. Light beam 342c is redirected by angled mirror 336 and angled mirror 338 to be collocated with light beam 342b. Light beams 342b and 342c are now directed by angled mirror 315 through lens 275, and onto optical fiber input 280.

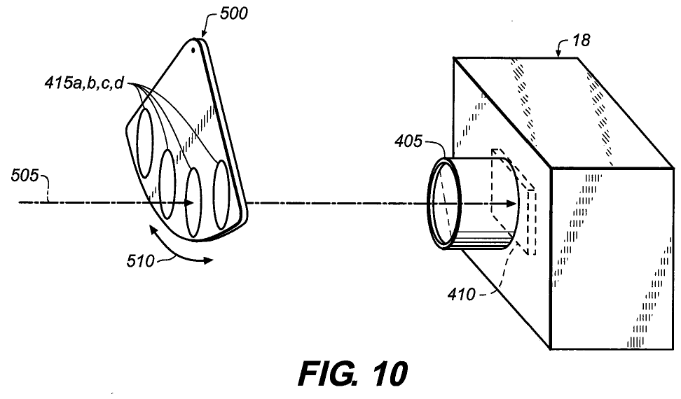

In the lens/camera system 18 shown in FIG. 10, the light 505 coming from an object plane 400 of the fluorescent image of the subject or object on sample object stage 104 is imaged by a camera lens 405, which may be a zoom lens with 10× variation in magnification, onto an electronic sensor 410 of lens and camera system 18. Light 505 is incident on a bandpass filter 415a, b, c, or d placed in front of camera lens 405. The bandpass filters 415a, b, c, or d may be of the type disclosed in U.S. Pat. No. 7,031,084.

In all of the aforementioned tuning of wavelength by tilting an interference filter, the effectiveness of a desired rejection or transmission is measured by the extent to which a desired wavelength is transmitted or an undesired wavelength is rejected. The efficacy with which the methodology meets a desirable endpoint depends upon the extent of light management needed to support a fluorescent imaging application peculiar to the application, and must be determined by those familiar to the art of so doing.

In one embodiment, a rotatable filter assembly 500, shown in FIG. 10, may be rotatably mounted and houses a plurality of different bandpass filters 415a, b, c, and d designed to pass specific wavelengths of transmitted light 505 coming from the object plane 400. The communication/computer control system 20 positions the appropriate bandpass filter 415a, b, c, and d into the path of light beam 505 as indicated by arrow 510 depending on transmitted wavelength of light emanating from the subject/object 112 on the sample object stage 104. Filter assembly 500 is designed to work in conjunction with spectral filter pack 230 and/or AOTF 290, each controlled and positioned by communication/computer control system 20 depending on the type of imaging probe used.

The apparatus of FIGS. 8 to 10 is useful for multimodal imaging of animals or tissues that have been treated with one or more of several types of imaging probes, typically by injection. Such imaging probes may comprise nanoparticles for use as carriers for bio-conjugation and targeted delivery which are stable so that they can be injected in vivo, especially intravascularly into an object to be placed on stage 104.

A variety of nanoparticles are useful in imaging probes suitable for imaging in accordance with the present invention. In multimodal imaging probes, the nanoparticle preferably has one or more targeting moieties and one or more diagnostic imaging components capable of being imaged by one or more imaging modes such as luminescence or fluorescent imaging component, X-ray and MRI.

As a first example, discussed subsequently in further detail, nanoparticles may be used that have the form of a nanolatex nanogel comprising a water-compatible, swollen, branched polymer network of repetitive, cross-linked, ethylenically unsaturated monomers as described in commonly assigned, copending U.S. patent application Ser. No. 11/732,424 filed Apr. 3, 2007 by Leon et al entitled “LOADED LATEX OPTICAL MOLECULAR IMAGING PROBES.” For an imaging probe using such a nanolatex nanogel particle to be multimodal the nanoparticle making up the probe must carry two or more imaging components, for example a near IR dye for fluorescent imaging and gadolinium for x-ray imaging.

As a further example, discussed subsequently in further detail, nanoparticles may be used that are derived from self-assembly of amphiphilic block or graft copolymers to form crosslink particles with imaging dye immobilized in the particle via covalent chemical bond in the core of the nanoparticles and alkoxy silane cross-linking resulting in organic/inorganic hybrid materials as described in commonly assigned, copending U.S. patent application Ser. No. 11/738,558 filed Apr. 23, 2007 by Shiying Zheng et al entitled “IMAGING CONTRAST AGENTS USING NANOPARTICLES.” In such nanoparticles derived from self-assembly, the imaging dyes contain functional groups that can react with the cross-linkable groups of the hydrophobic component and are immobilized in the core of the nanoparticles by covalent bonding. More specifically the imaging dyes contain alkoxy silane groups. Since the imaging dyes are immobilized in the nanoparticles, the quantum efficiency is enhanced.

As another example, discussed subsequently in detail, an amine-modified silica nanoparticle having a polymer shell comprising amine functionalities may be used as described in commonly assigned, copending U.S. patent application Ser. No. 11/872,866 filed Oct. 16, 2007 by Zheng et al entitled “SILICA-CORED CARRIER PARTICLE” and its Continuation application Ser. No. 11/930,417 filed Oct. 31, 2007 by Zheng et al. The core/shell particle has attached one or more fluorescent groups, polymer groups such as polyethylene glycol, targeting molecules, antibodies or peptides. Suitable particles are described in the U.S. Patent Application of Bringley et al, previously mentioned. Especially useful are silica nanoparticles having a near infrared fluorescent core and having attached to their surface, amine groups and/or polyethylene glycol. Suitable particles also are described in the U.S. Patent Application of Bringley et al.

Such exemplary nanoparticles have been found to be nontoxic, and are capable of entry into small capillaries in the body, transport in the body to a disease site, crossing biological barriers (including but not limited to the blood-brain barrier and intestinal epithelium), absorption into cell endocytic vesicles, crossing cell membranes and transportation to the target site inside the cell. The particles in that size range are believed to be efficiently transferred across the arterial wall compared to larger size microparticles, see Labhasetwar et al., previously cited. Without being bound by any particular theory, it is also believed that because of high surface to volume ratio, the small size provides successful targeting of such particles using targeting molecules.

Whenever used in the specification the terms set forth shall have the following meaning:

The term “nanoparticle” or “nanoparticulate” refers to a particle with a size of less than 100 nm.

The term “colloid” refers to a mixture of small particulates dispersed in a liquid, such as water.

The term “biocompatible” means that a composition does not disrupt the normal function of the bio-system into which it is introduced. Typically, a biocompatible composition will be compatible with blood and does not otherwise cause an adverse reaction in the body. For example, to be biocompatible, the material should not be toxic, immunogenic or thrombogenic.

The term “biodegradable” means that the material can be degraded either enzymatically or hydrolytically under physiological conditions to smaller molecules that can be eliminated from the body through normal processes.

The term “targeting molecule” refers to any molecule, atom, or ion linked to the polymer networks or surface of the current invention that enhance binding, transport, accumulation, residence time, bioavailability or modify biological activity of the polymer networks or biologically active compositions of the current invention in the body or cell. The targeting molecule will frequently comprise an antibody, fragment of antibody or chimeric antibody molecules typically with specificity for a certain cell surface antigen. It could also be, for instance, a hormone having a specific interaction with a cell surface receptor, or a drug having a cell surface receptor. For example, glycolipids could serve to target a polysaccharide receptor. It could also be, for instance, enzymes, lectins, and polysaccharides. Low molecular mass ligands, such as folic acid and derivatives thereof are also useful in the context of the current invention. The targeting molecules can also be polynucleotide, polypeptide, peptidomimetic, carbohydrates including polysaccharides, derivatives thereof or other chemical entities obtained by means of combinatorial chemistry and biology. Targeting molecules can be used to facilitate intracellular transport of the nanoparticles of the invention, for instance transport to the nucleus, by using, for example, fusogenic peptides as targeting molecules described by Soukchareun et al., Bioconjugate Chem., 6, 43, (1995) or Arar et al., Bioconjugate Chem., 6, 43 (1995), caryotypic peptides, or other biospecific groups providing site-directed transport into a cell (in particular, exit from endosomic compartments into cytoplasm, or delivery to the nucleus).

The described composition can comprise a biological, pharmaceutical or diagnostic component that includes a targeting moiety that recognizes the specific target cell. Recognition and binding of a cell surface receptor through a targeting moiety associated with a described nanoparticle used as a carrier can be a feature of the described compositions. For purposes of the present invention, a compound carried by the nanoparticle may be referred to as a “carried” compound. For example, the biological, pharmaceutical or diagnostic component that includes a targeting moiety that recognizes the specific target cell described above is a “carried” compound. This feature takes advantage of the understanding that a cell surface binding event is often the initiating step in a cellular cascade leading to a range of events, notably receptor-mediated endocytosis. The term “Receptor Mediated Endocytosis” (“RME”) generally describes a mechanism by which, catalyzed by the binding of a ligand to a receptor disposed on the surface of a cell, a receptor-bound ligand is internalized within a cell. Many proteins and other structures enter cells via receptor mediated endocytosis, including insulin, epidermal growth factor, growth hormone, thyroid stimulating hormone, nerve growth factor, calcitonin, glucagon and many others.

Receptor Mediated Endocytosis affords a convenient mechanism for transporting a described nanoparticle, possibly containing other biological, pharmaceutical or diagnostic components, to the interior of a cell. In RME, the binding of a ligand by a receptor disposed on the surface of a cell can initiate an intracellular signal, which can include an endocytosis response. Thus, a nanoparticle used as a carrier with an associated targeting moiety, can bind on the surface of a cell and subsequently be invaginated and internalized within the cell. A representative, but non-limiting, list of moieties that can be employed as targeting agents useful with the present compositions includes proteins, peptides, aptomers, small organic molecules, toxins, diphtheria toxin, pseudomonas toxin, cholera toxin, ricin, concanavalin A, Rous sarcoma virus, Semliki forest virus, vesicular stomatitis virus, adenovirus, transferrin, low density lipoprotein, transcobalamin, yolk proteins, epidermal growth factor, growth hormone, thyroid stimulating hormone, nerve growth factor, calcitonin, glucagon, prolactin, luteinizing hormone, thyroid hormone, platelet derived growth factor, interferon, catecholamines, peptidomimetrics, glycolipids, glycoproteins and polysaccharides. Homologs or fragments of the presented moieties can also be employed. These targeting moieties can be associated with a nanoparticle and be used to direct the nanoparticle to a target cell, where it can subsequently be internalized. There is no requirement that the entire moiety be used as a targeting moiety. Smaller fragments of these moieties known to interact with a specific receptor or other structure can also be used as a targeting moiety.

An antibody or an antibody fragment represents a class of most universally used targeting moiety that can be utilized to enhance the uptake of nanoparticles into a cell. Antibodies may be prepared by any of a variety of techniques known to those of ordinary skill in the art. See, e.g., Harlow and Lane, Antibodies: A Laboratory Manual, Cold Spring Harbor Laboratory, 1988. Antibodies can be produced by cell culture techniques, including the generation of monoclonal antibodies or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). A superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described Winter et al. (1991) Nature 349:293-299; Lobuglio et al. (1989) Proc. Nat. Acad. Sci. USA 86:4220-4224. These “humanized” molecules are designed to minimize unwanted immunological response toward rodent antihuman antibody molecules that limits the duration and effectiveness of therapeutic applications of those moieties in human recipients.

Vitamins and other essential minerals and nutrients can be utilized as targeting moiety to enhance the uptake of nanoparticle by a cell. In particular, a vitamin ligand can be selected from the group consisting of folate, folate receptor-binding analogs of folate, and other folate receptor-binding ligands, biotin, biotin receptor-binding analogs of biotin and other biotin receptor-binding ligands, riboflavin, riboflavin receptor-binding analogs of riboflavin and other riboflavin receptor-binding ligands, and thiamin, thiamin receptor-binding analogs of thiamin and other thiamin receptor-binding ligands. Additional nutrients believed to trigger receptor mediated endocytosis, and thus also having application in accordance with the presently disclosed method, are carnitine, inositol, lipoic acid, niacin, pantothenic acid, pyridoxal, and ascorbic acid, and the lipid soluble vitamins A, D, E and K. Furthermore, any of the “immunoliposomes” (liposomes having an antibody linked to the surface of the liposome) described in the prior art are suitable for use with the described compositions.

Since not all natural cell membranes possess biologically active biotin or folate receptors, use of the described compositions in-vitro on a particular cell line can involve altering or otherwise modifying that cell line first to ensure the presence of biologically active biotin or folate receptors. Thus, the number of biotin or folate receptors on a cell membrane can be increased by growing a cell line on biotin or folate deficient substrates to promote biotin and folate receptor production, or by expression of an inserted foreign gene for the protein or apoprotein corresponding to the biotin or folate receptor.

RME is not the exclusive method by which the described nanoparticle can be translocated into a cell. Other methods of uptake that can be exploited by attaching the appropriate entity to a nanoparticle include the advantageous use of membrane pores. Phagocytotic and pinocytotic mechanisms also offer advantageous mechanisms by which a nanoparticle can be internalized inside a cell.

The recognition moiety can further comprise a sequence that is subject to enzymatic or electrochemical cleavage. The recognition moiety can thus comprise a sequence that is susceptible to cleavage by enzymes present at various locations inside a cell, such as proteases or restriction endonucleases (e.g. DNAse or RNAse).

A cell surface recognition sequence is not a requirement. Thus, although a cell surface receptor targeting moiety can be useful for targeting a given cell type, or for inducing the association of a described nanoparticle with a cell surface, there is no requirement that a cell surface receptor targeting moiety be present on the surface of a nanoparticle.

To assemble the biological, pharmaceutical or diagnostic components to a described nanoparticle used as a carrier, the components can be associated with the nanoparticle carrier through a linkage. By “associated with”, it is meant that the component is carried by the nanoparticle. The component can be dissolved and incorporated in the nanoparticle non-covalently.

Generally, any manner of forming a linkage between a biological, pharmaceutical or diagnostic component of interest and a nanoparticle used as a carrier can be utilized. This can include covalent, ionic, or hydrogen bonding of the ligand to the exogenous molecule, either directly or indirectly via a linking group. The linkage is typically formed by covalent bonding of the biological, pharmaceutical or diagnostic component to the nanoparticle used as a carrier through the formation of amide, ester or imino bonds between acid, aldehyde, hydroxy, amino, or hydrozoa groups on the respective components of the complex. Art-recognized biologically labile covalent linkages such as imino bonds and so-called “active” esters having the linkage —COOCH, —O—O— or —COOCH are preferred. The biological, pharmaceutical or diagnostic component of interest may be attached to the pre-formed nanoparticle or alternately the component of interest may be pre-attached to a polymerizeable unit and polymerized directly into the nanoparticle during the nanoparticle preparation. Hydrogen bonding, e.g., that occurring between complementary strands of nucleic acids, can also be used for linkage formation.

In the imaging probe as described in the previously mentioned U.S. patent application Ser. No. 11/732,424, the nanoparticle may be in the form of a nanolatex nanogel comprising a water-compatible, swollen, branched polymer network of repetitive, crosslinked, ethylenically unsaturated monomers of Formula I:

(X)m—(Y)n-(Z)o Formula I

wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional cross-linking monomer; m ranges from 50-90 mol %; n ranges from 2-30 mol %; and o range from 1-15 mol %. The present invention also relates to a method for preparing a nanogel comprising preparing a header composition of a mixture of monomers X, Y, and Z, and a first portion of initiators in water, wherein X is a water-soluble monomer containing ionic or hydrogen bonding moieties, Y is a water-soluble macromonomer containing repetitive hydrophilic units bound to a polymerizeable ethylenically unsaturated group, and Z is a multifunctional cross-linking monomer; preparing a reactor composition of a second portion initiators, surfactant, and water sufficient to afford a composition of 1-10% w/w of monomers X, Y, and Z; bringing the reactor composition to the polymerization temperature; holding the reactor composition at the polymerization temperature for the duration of the reaction, and adding the header composition to the reactor composition over time to form a reaction mixture, wherein the nanogel comprises a water-compatible, swollen, branched polymer network of repetitive, crosslinked, ethylenically unsaturated monomers of Formula I:

(X)m—(Y)n-(Z)o Formula I

wherein m ranges from 50-90 mol %; n ranges from 2-30 mol %; and o range from 1-15 mol %. For the imaging probe to be multimodal the nanoparticle making up the probe must carry two or more imaging components for example a near IR dye for fluorescent imaging and gadolinium for x-ray imaging.

In the imaging probe as described in the previously mentioned U.S. patent application Ser. No. 11/738,558, the nanoparticles are derived from self-assembly of amphiphilic block or graft copolymers to form crosslink particles with imaging dye immobilized in the particle, more specifically the imaging dye is immobilized via covalent chemical bond in the core of the nanoparticles and alkoxy silane cross-linking results in organic/inorganic hybrid materials.

It is well known that, in the presence of a solvent or solvent mixture that is selective for on block, amphiphilic block or graft copolymers have the ability to assemble into colloidal aggregates of various morphologies. In particular, significant interest has been focused on the formation of polymeric micelles and nanoparticles from amphiphilic block or graft copolymers in aqueous media. This organized association occurs as polymer chains reorganize to minimize interactions between the insoluble hydrophobic blocks and water. The resulting nanoparticles possess cores composed of hydrophobic block segments surrounded by outer shells of hydrophilic block segments. The core-shell structures of amphiphilic micellar assemblies have been utilized as carrier systems in the filed of drug delivery.

The amphiphilic copolymers that are useful in the present invention have a hydrophilic water soluble component and a hydrophobic component. Useful water soluble components include poly(alkylene oxide), poly(saccharides), dextrans, and poly(2-ethyloxazolines), preferably poly(ethylene oxide). Hydrophobic components useful in the present invention include but are not limited to styrenics, acrylamides, (meth)acrylates, lactones, lactic acid, and amino acids. Preferably, the hydrophobic components derived from styrenics and (meth)acrylates containing cross-linkable alkoxy silane groups. The imaging dyes contain functional groups that can react with the cross-linkable groups of the hydrophobic component and are immobilized in the core of the nanoparticles by covalent bonding. More specifically the imaging dyes contain alkoxy silane groups. Since the imaging dyes are immobilized in the nanoparticles, the quantum efficiency is enhanced. Suitable particles are described in the U.S. Patent Application of Zheng et al, previously mentioned.

In the imaging probe as described in the previously mentioned U.S. patent applications Ser. No. 11,872,866 and Ser. No. 11/930,417, the nanoparticle may be in the form of an amine-modified silica nanoparticle, having a polymer shell comprising amine functionalities. The core/shell particle has attached one or more fluorescent groups, polymer groups such as polyethylene glycol, targeting molecules, antibodies or peptides. Suitable particles are described in the U.S. Patent Application of Bringley et al, previously mentioned. Especially preferred are silica nanoparticles having a near infrared fluorescent core and having attached to their surface, amine groups and/or polyethylene glycol. Suitable particles also are described in the U.S. Patent Application of Bringley et al, previously mentioned.

In multimodal imaging probes the nanoparticle has one or more imaging components capable of being imaged by one or more imaging modes such as luminescence or fluorescent imaging component, X-ray and MRI.

The luminescence or fluorescent imaging component can be a near IR dye. Fluorophores include organic, inorganic or metallic materials that luminesce with including phosphorescence, fluorescence and chemo luminescence and bioluminescence. Examples of fluorophores include organic dyes such as those belonging to the class of naphthalocyanines, phthalocyanines, porphyrins, coumarins, oxanols, flouresceins, rhodamines, cyanines, dipyrromethanes, azadipyrromethanes, squaraines, phenoxazines; metals which include gold, cadmium selenides, cadmium telerides; and proteins such as green fluorescent protein and phycobiliprotein, and chemo luminescence by oxidation of luminal, substituted benzidines, substituted carbazoles, substituted naphthols, substituted benzthiazolines, and substituted acridans.

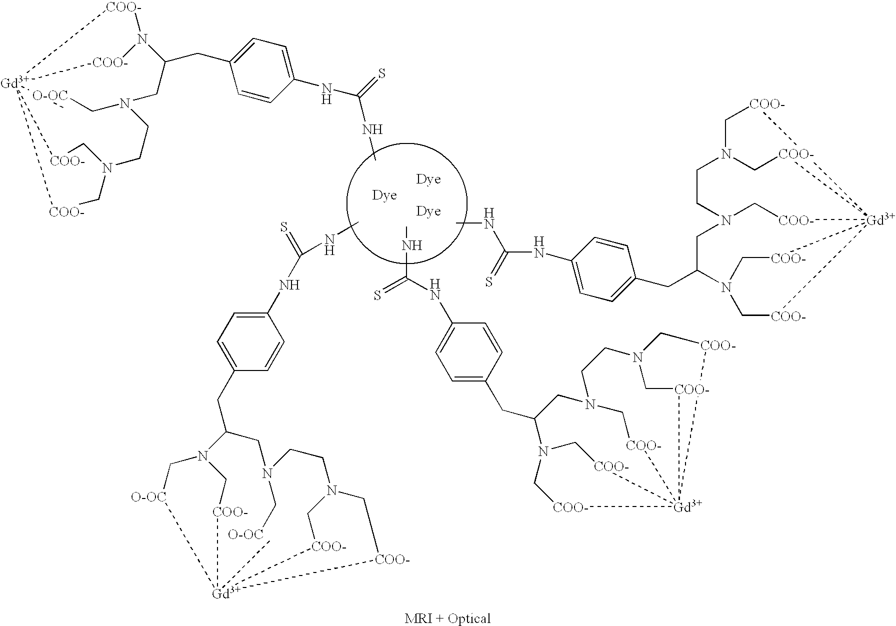

Where Dye is represented by the structure

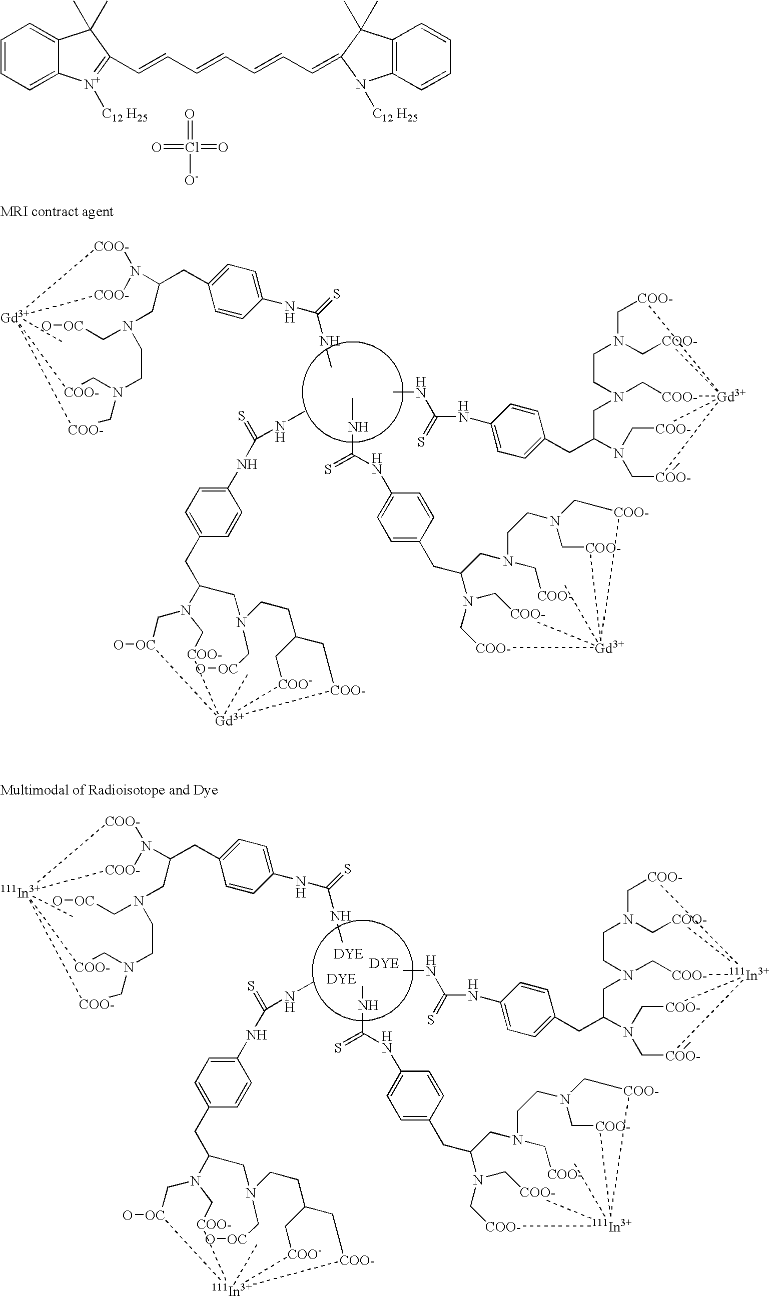

Where dye is represented by

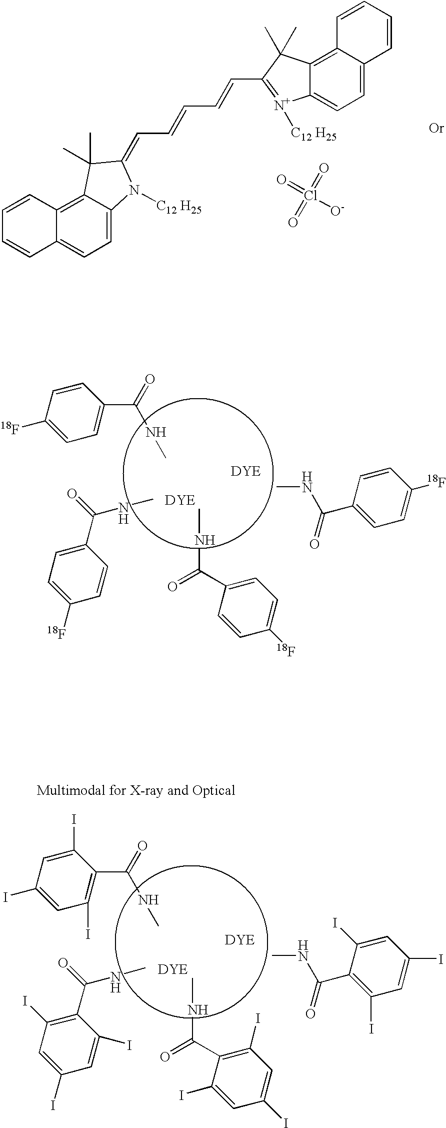

Where dye is represented by:

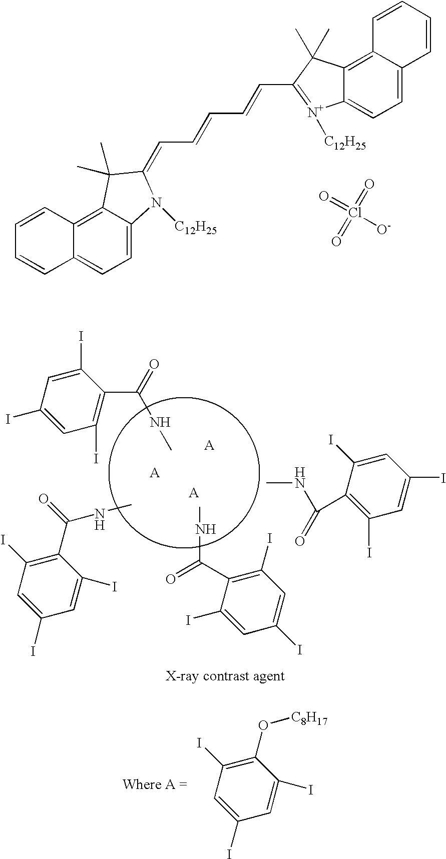

The invention has been described in detail with particular reference to certain preferred embodiments thereof, but it will be understood that variations and modifications can be effected within the spirit and scope of the invention.

PARTS LIST

| | |
|---|---|
| 10 | electronic imaging system |
| 12 | light source |
| 14 | optical compartment |
| 16 | mirror |
| 18 | lens/camera system |
| 20 | communication/computer |
| | control system |
| 100 | imaging system of the |
| | present invention |
| 102 | x-ray source |
| 104 | sample object support stage |
| 106 | epi-illumination; fiber optics |
| 108 | sample environment |
| 110 | access means/member |
| 112 | subject/object |
| 120 | frame |
| 122 | support sheet |
| 125 | phosphor plate |
| 126 | optical platen |
| 128 | protective layer |
| 130 | phosphor layer |
| 132 | optical layer |
| 134 | support plate; glass |
| 136 | air gap/void |
| 138 | air gap/void |
| 200 | programmable multispectral |
| | light source |
| 205 | xenon lamp |
| 210 | hot mirror |
| 215 | lens |
| 220 | fold mirror |
| 222 | aperture |
| 225 | collimating lens |
| 230 | bandpass interference filter |
| | assembly |
| 235a, b, c, d | bandpass |
| | interference filters |
| 240 | beam of light |
| 240a | bandpass filtered light |
| | beam |
| 240b | −1^{st} diffracted order filtered |
| | light beam |
| 240c | +1^{st} diffracted order filtered |
| | light beam |
| 245 | arrow showing movement of |
| | assembly 230 |
| 248 | arrow showing movement of |
| | assembly 250 |
| 250 | movable light path control |
| | assembly |
| 255 | blank hole |
| 260 | first angled mirror |
| 265 | second angled mirror |
| 275 | lens |
| 280 | optical fiber input |
| 281 | bandpass interference filters |
| 282 | wheel |
| 283 | arrow showing movement of |
| | wheel 282 |
| 284 | arrow showing movement of |
| | wheel 282 |
| 285 | third angled mirror |
| 286 | tilting mechanism |
| 287 | arrow showing movement of |
| | spectral filter assembly 230 |
| 288 | axle for tilting assembly 230 |
| 290 | acousto-optical tunable filter |
| 295 | fourth angled mirror |
| 300 | baffling wall |
| 301 | fifth angled mirror |
| 302 | sixth angled mirror |
| 305 | seventh angled mirror |
| 310 | undiffracted 0^{th} order light |
| | beam |
| 315 | eighth angled mirror |
| 316 | low-pass interference filter |
| | assembly |
| 316a, b, c and d | low-pass |
| | interference filters |
| 318 | low-pass filtered light beam |
| 318b | −1^{st} diffracted order |
| | doubly-filtered light beam |
| 318c | +1^{st} diffracted order |
| | doubly-filtered light beam |
| 320 | undiffracted 0^{th} order light |
| | beam |
| 330 | acousto-optical tunable filter |
| 332 | angled mirror |
| 334 | baffling wall |
| 336 | angled mirror |
| 338 | angled mirror |
| 340 | residual undiffracted 0^{th} |
| | order light beam |
| 342b | −1^{st} diffracted order |
| | doubly-filtered light beam |
| 342c | +1^{st} diffracted order |
| | doubly-filtered light beam |
| 400 | object plane |
| 405 | camera lens |
| 410 | electronic sensor |
| 415a, b, c, d | bandpass filters |
| 500 | filter assembly |
| 505 | transmitted light |
| 510 | arrow showing movement of |
| | assembly 500 |

What is claimed is:

1. An apparatus for multimodal imaging of an object, comprising:
a support stage for receiving an object to be imaged;
an object supported on the stage;
a light source for producing a beam to illuminate the object;
a filter positioned to receive and pass the beam toward the object; the filter comprising a first acousto-optical tunable filter (AOTF), the beam being substantially unpolarized as incident on the AOTF; and
a lens and camera system for capturing a first image of the object.

2. An apparatus according to claim 1, wherein the AOTF is tiltable relative to the beam for aligning the AOTF to a desired angle of incidence.

3. An apparatus according to claim 1, further comprising baffling walls for eliminating undesired wavelength light from an output of the AOTF.

4. An apparatus according to claim 3, wherein the light beam from the source has a divergence half-angle and the AOTF has a beam separation angle, the divergence half-angle and the divergence angle being chosen to cause spatial separation of the undiffracted 0^{th} order, +1^{st} diffracted order, and −1^{st} order diffracted beams of the AOTF, where by the baffling walls are effective to remove the undiffracted 0^{th} order beam from light to illuminate the object.

5. An apparatus according to claim 9, further comprising, between the light source and the AOTF, a beam expander lens, a collimating lens and a spatial filter located at the common focus of the beam expander and collimating lenses.

6. An apparatus according to claim 1, further comprising a second AOTF cascaded with the first AOTF, whereby the beam is doubly filtered.

7. An apparatus according to claim 1, further comprising an interference filter cascaded with the AOTF.

8. An apparatus according to claim 7 in which the interference filter is one in which tilt relative to the beam affects the spectral band that the interference filter passes, further comprising means for tilting the filter relative to the beam.

9. An apparatus for multimodal imaging of an object, comprising:
a light source;
a support stage for receiving an object to be imaged;
means for selectively directing light from the light source through a first filter assembly to produce a first beam of light of a first frequency range for illuminating an object on the stage or through a second acousto-optical tunable filter (AOTF) to produce a second beam of light of a second frequency range for illuminating an object on the stage; and
a lens and camera system for capturing light from the object illuminated by either the first or second beam of light to produce a first image in response to the first beam and a second image, different from the first image, in response to the second beam.

10. An apparatus according to claim 9, wherein the means for selectively directing comprises a movable light path control assembly having optical elements for directing light from the source into either the first beam or the second beam.

11. An apparatus according to claim 10, wherein the means for selectively directing comprises a first filter for passing light in the first frequency range and a second filter for passing light in the second frequency range.

12. An apparatus according to claim 11, further comprising a third filter assembly for filtering light directed to the lens and camera system.

13. An apparatus according to claim 11 wherein the first and second filters are tiltable relative to their respective beams.

14. An apparatus according to claim 9, where in the first filter is slideably mounted.

15. An apparatus according to claim 14, wherein the first filter is tiltable relative to incident light.

16. An apparatus according to claim 9, wherein the first filter is rotatably mounted.

17. An apparatus according to claim 16, wherein the first filter is tiltable relative to incident light.
